(19) United States
(12) Patent Application Publication
(10) Pub. No.: US 2017/0002398 A1
(43) Pub. Date: Jan. 5, 2017

(54) NUCLEIC ACID AMPLIFICATION METHOD

(71) Applicant: QIAGEN GMBH, Hilden (DE)

(72) Inventors: Daniel Muller, Hilden (DE); Marlo Scherer, Solingen (DE); Harald Quintel, Steckborn (CH)

(21) Appl. No.: 15/113,308

(22) PCT Filed: Jan. 7, 2015
(86) PCT No.: PCT/EP2015/050131

§ 371 Date: Jul. 21, 2016

(30) Foreign Application Priority Data
Jan. 23, 2014 (EP) 14152306.8

(51) Int. Cl. C12Q 1/68

(52) U.S. Cl. CPC C12Q 1/686

(57) ABSTRACT

The invention relates to a method for the simultaneous detection of at least four nucleic acids labeled with a covalently attached dye in a sample comprising the step of detecting the fluorescence emission of said fluorescent dyes upon excitation, wherein the at least four dyes attached to said nucleic acids are selected from the groups of i) 6-FAM or 5-FAM or a blend thereof covalently attached to a first nucleic acid, ii) DY-530, HEX or ATTO 532 or a blend thereof covalently attached to a second nucleic acid, iii) ATT0550 or DY-555 or a blend thereof covalently attached to a third nucleic acid, iv) ATT0565, DY510-XL or ROX or a blend thereof covalently attached to a fourth nucleic acid, v) DY632 or DY520-XL or a blend thereof covalently attached to a fifth nucleic acid and, vi) Chromeo 494 attached to a sixth nucleic acid, wherein Chromeo 494 must be one of the four selected.

### FIELD OF THE INVENTION

The present invention is in the field of molecular biology. Particularly, the present invention relates to the detection of nucleic acids, e.g. in amplification reactions such as polymerase chain reactions (PCR). The present invention is particularly useful in genotyping, quantitative PCR, real-time PCR and multiplex-PCR. The present invention particularly relates to dyes used in PCR.

### BACKGROUND

Fluorescent dyes have a wide application in analytical chemistry, biochemistry and molecular biology, such as in DNA sequencing and during detection and amplification of nucleic acids. Fluorescent dyes absorb light of a specific wavelength (“excitation”) and re-emit energy at a different specific wavelength (“emission”). The fluorescent dyes are frequently used as molecular labels attached to probes or other biomolecules.

Multiplex polymerase chain reactions (multiplex PCR) is a PCR technique that enables amplification of two or more products in parallel in a single reaction tube. It is widely used in genotyping applications and different areas of molecular biology, e.g. in research, forensic and diagnostic applications, including human identification and paternity testing and for diagnosis of infectious diseases or chimerism analysis after allogeneic bone marrow transplantation. Multiplex PCR can also be used for qualitative and semi-quantitative gene expression analysis using cDNA as a starting template or in combination with reverse transcription with mRNA as starting material. The nucleic acids analyzed may for example originate from a variety of eukaryotic (human, animal or plant) and prokaryotic (bacterial) or viral sources.

Multiplex PCR is for example used for the simultaneous detection of multiple marker genes and/or their polymorphisms, e.g. short tandem repeats (STRs) or deletion insertion polymorphisms (DIPS or Indels). Detection of the amplification products and their genotyping is usually carried out by multiple color fluorescence detection after electrophorectic separation (e.g. capillary gel electrophoresis) in DNA sequencers. In real-time, quantitative multiplex PCR, the amplification of multiple target sequences can be monitored at the same time by simultaneous detection of fluorescence of different fluorescent dyes.

For the purpose of multiple colour fluorescence detection, at least one primer of each primer pair used during amplification is labeled by covalently bond fluorescent dye at is 5′-OH end or at an internal base.

To date, the simultaneous amplification of 8 to 16 or even more target sequences is possible. However, modern thermocyclers (for real-time PCR) and DNA sequencers used in multiplex PCR are adapted for the simultaneous detection of 3 to 5 different fluorescent dyes. Usually one of the fluorescent dyes is used as a label of a length standard when gel electrophoresis analysis is performed.

The apparatuses used for detection are often designed and calibrated for the use of combinations of very specific fluorescent dyes, i.e. in terms of excitation and detection wavelengths and filters used. The optical set-up of many devices encompasses the use of one Argon laser (488 nm) or a solid state diode (505 nm), a spectrograph, virtual filters, in combination with a CCD camera and algorithms for spectral calculation. This set-up severely limits the amount of dyes that may be used.

At the same time the identification of a dye in the spectral emission region 605 to 630 nm is difficult. These are difficult to excite by the known and used lasers. However the sensitivity in this range is the relevant factor for the overall sensitivity of the set-up. Hence, it would be desirable to have an ideal dye also in this spectral range.

### BRIEF DESCRIPTION OF THE INVENTION

The present invention relates to specific combinations of fluorescent dyes for the detection of fluorescently labeled nucleic acids.

The invention relates to a method for the simultaneous detection of at least four nucleic acids labeled with a covalently attached dye in a sample comprising the step of:
detecting the fluorescence emission of said fluorescent dyes upon excitation, wherein the at least four dyes attached to said nucleic acids are selected from the groups of:
   i. 6-FAM or 5-FAM or a blend thereof covalently attached to a first nucleic acid,
   ii. DY-530, HEX or ATTO 532 or a blend thereof covalently attached to a second nucleic acid,
   iii. ATT0550 or DY-555 or a blend thereof covalently attached to a third nucleic acid,
   iv. ATT0565, DY510-XL or ROX or a blend thereof covalently attached to a fourth nucleic acid,
   v. DY632 or DY520-XL or a blend thereof covalently attached covalently attached to a fifth nucleic acid and,
   vi. Chromeo 494 attached to a sixth nucleic acid, wherein
      Chromeo 494 must be one of the four selected

The invention also relates to a method for the simultaneous detection of at least four amplification products from an amplification reaction, comprising the steps of
(i) amplifying three or more loci on nucleic acid templates using primers or pairs of primers substantially complementary to sequences flanking said loci; and
(ii) detecting said amplification products by fluorescence detection of fluorescently labelled amplification products or alternatively by fluorescence detection of fluorescently labelled oligonucleotide probes complementary to sequences on said loci of said amplification products,
(iii) wherein a fluorescent dye is covalently attached to said amplification product and/or to said oligonucleotide probe and/or to said primer and/or to at least one primer of said primer pair and optionally to a size marker,
(iv) and wherein at least four different dyes are used for differentiating the at least four amplification products wherein, the dyes may be selected from the group comprising:
   i. a first dye which is 6-FAM or 5-FAM or a blend thereof,
   ii. a second dye which is DY-530, HEX or ATTO 532 or a blend thereof,
   iii. a third dye which is ATT0550 or DY-555 or a blend thereof,
   iv. a fourth dye which is ATT0565, DY-510XL or ROX or a blend thereof,
   v. a fifth dye which is DY632 or DY520-XL or a blend thereof,
   vi. and a sixth dye which is Chromeo 494 and, wherein
      one dye must be Chromeo 494.

And, the invention relates to a kit for multiplex PCR or PCR comprising at least four different fluorescent dyes suitable for the covalent attachment to nucleotides, primers, size markers or oligonucleotide probes used in multiplex PCR, wherein the four dyes may be selected from the six groups below:
i. 6-FAM or 5-FAM or a blend thereof,
ii. DY-530, HEX or ATTO 532 or a blend thereof,
iii. ATT0550 or DY-555 or a blend thereof,
iv. ATT0565, DY-510XL or ROX or a blend thereof covalently, or
v. DY632, and
vi. Chromeo 494, wherein
one dye must be Chromeo 494.

### DETAILED DESCRIPTION OF THE INVENTION

The dye collection described herein for the first time provides for higher sensitivity, less cross talk between color channels and compatibility with numerous existing devices.

The present invention relates to specific combinations of fluorescent dyes for the detection of fluorescently labeled nucleic acids. The structures of 5-FAM (5-carboxyfluorescin) and 6-FAM (6-carboxyfluorescin) are illustrated in the figures. The structures of HEX (6-carboxy-2′,4,4′,5′,7,7′-hexachlorofluorescein) and ROX (5-carboxy-X-rhodamine) are illustrated in the figures. The structures of DY-530, DY-555, DY-556, DY-510XL, DY-632 and DY-520XL are illustrated in the figures. The structures of ATTO 532, ATTO 550 and ATTO 565 are illustrated in the figures. DY-530, DY-555, DY-556, DY-510XL, DY-632 and DY-520XL are products of Dyomics GmbH, Jena, Germany. ATTO 532, ATTO 550 and ATTO 565 are products of ATTO-TEC GMBH, Siegen, Germany. Chromeo™ 494 is a product of Active Motif, Carlsbad, Calif., US.

The invention relates to a method for the simultaneous detection of at least four nucleic acids labeled with a covalently attached dye in a sample comprising the step of:
detecting the fluorescence emission of said fluorescent dyes upon excitation, wherein the at least four dyes attached to said nucleic acids are selected from the groups of:
   i. 6-FAM or 5-FAM or a blend thereof covalently attached to a first nucleic acid,
   ii. DY-530, HEX or ATTO 532 or a blend thereof covalently attached to a second nucleic acid,
   iii. ATT0550 or DY-555 or a blend thereof covalently attached to a third nucleic acid,
   iv. ATT0565, DY-510XL or ROX or a blend thereof covalently attached to a fourth nucleic acid,
   v. DY632 or DY520-XL or a blend thereof covalently attached to a fifth nucleic acid and,
   vi. Chromeo 494 attached to a sixth nucleic acid, wherein
      Chromeo 494 must be one of the four selected

In a preferred embodiment, the invention relates to a method for the simultaneous detection of at least six nucleic acids labeled with a covalently attached dye in a sample comprising the step of:
detecting the fluorescence emission of said fluorescent dyes upon excitation, wherein the at least six dyes attached to said nucleic acids are selected from the groups of:
   i. 6-FAM or 5-FAM or a blend thereof covalently attached to a nucleic acid,
   ii. DY-530, HEX or ATTO 532 or a blend thereof covalently attached to a nucleic acid,
   iii. ATT0550 or DY-555 or a blend thereof covalently attached to a nucleic acid,
   iv. ATT0565, DY510-XL or ROX or a blend thereof covalently attached to a nucleic acid,
   v. DY632 or DY520-XL or a blend thereof covalently attached to a nucleic acid and,
   vi. Chromeo 494 attached to a nucleic acid.

The invention also relates to a method for the simultaneous detection of at least four amplification products from an amplification reaction, comprising the steps of
(i) amplifying three or more loci on nucleic acid templates using primers or pairs of primers substantially complementary to sequences flanking said loci; and
(ii) detecting said amplification products by fluorescence detection of fluorescently labelled amplification products or alternatively by fluorescence detection of fluorescently labelled oligonucleotide probes complementary to sequences on said loci of said amplification products,
(iii) wherein a fluorescent dye is covalently attached to said amplification product and/or to said oligonucleotide probe and/or to said primer and/or to at least one primer of said primer pair and optionally to a size marker,
(iv) and wherein at least four different dyes are used for differentiating the at least four amplification products wherein, the dyes may be selected from the group comprising:
   i. a first dye which is 6-FAM or 5-FAM or a blend thereof,
   ii. a second dye which is DY-530, HEX or ATTO 532 or a blend thereof,
   iii. a third dye which is ATT0550 or DY-555 or a blend thereof,
   iv. a fourth dye which is ATT0565, DY510-XL or ROX or a blend thereof covalently,
   v. a fifth dye which is DY632 or DY520-XL or a blend thereof,
   vi. and a sixth dye which is Chromeo 494 and, wherein
      one dye must be Chromeo 494.

In a preferred embodiment, the invention relates to a method for the simultaneous detection of at least six amplification products from an amplification reaction, comprising the steps of:
(i) amplifying five or more loci on nucleic acid templates using primers or pairs of primers substantially complementary to sequences flanking said loci; and
(ii) detecting said amplification products by fluorescence detection of fluorescently labelled amplification products or alternatively by fluorescence detection of fluorescently labelled oligonucleotide probes complementary to sequences on said loci of said amplification products,
(iii) wherein a fluorescent dye is covalently attached to said amplification product and/or to said oligonucleotide probe and/or to said primer and/or to at least one primer of said primer pair and optionally to a size marker,
(iv) and wherein at least six different dyes are used for differentiating the at least six amplification products wherein, the dyes may be selected from the group comprising:
   i. a first dye which is 6-FAM or 5-FAM or a blend thereof,
   ii. a second dye which is DY-530, HEX or ATTO 532 or a blend thereof,
   iii. a third dye which is ATT0550 or DY-555 or a blend thereof,
   iv. a fourth dye which is ATT0565, DY510-XL or ROX or a blend thereof,
   v. a fifth dye which is DY632 or DY520-XL or a blend thereof,
   vi. and a sixth dye which is Chromeo 494.

As outlined above, the invention relate to combinations of at least four different fluorescent dyes. In the context of the above described methods this means that when a size marker fluorescently labelled with one dye of the invention is used, at least three nucleic acid sequences (i.e. loci) can be amplified and detected, depending on whether a combination of four, five or even more different dyes is used. If no fluorescently labelled size marker is used, then at least four, five, or six sequences can be amplified and detected.

A “locus” in the context of the present invention is a part of the sequence of a nucleic acid, e.g. a chromosome, mRNA, plasmid, cosmid, DNA or RNA (of any origin, e.g. bacterial, viral, eukaryotic, prokaryotic, from plants, fungi or animals, e.g. of human origin) and the like, that is to be amplified and/or detected.

“Amplification products” herein are nucleic acids or oligonucleotides that are the product of an amplification reaction, e.g. of a polymerase chain reaction. They are for example defined by the primers used for amplification.

A “primer” herein refers to an oligonucleotide comprising a sequence that is complementary to a nucleic acid to be amplified or transcribed (“template”). During replication polymerases attach nucleotides to the 3′-OH end of the primer complementary to the respective nucleotides of the template.

An “oligonucleotide” herein refers to a stretch of nucleic acid, e.g. RNA or DNA, that comprises a sequence of two or more nucleotides, e.g. between 2 and 250 nucleotides, more preferably between 2 and 200, even more preferably between 2 and 100, even more preferably between 2 and 30, even more preferably between 2 and 25, even more preferably between 2 and 20, even more preferably between 5 and 25, and most preferably between 10 and 25 nucleotides.

In the first aspect of the method, during amplification detection may for example occur using fluorescently labelled oligonucleotide probes. Alternatively fluorescently labelled primers may be used. In another alternative, fluorescently labelled nucleotides may be used that are incorporated into the amplification products. In this case it is preferred that for every locus to be amplified or detected, the amplification is performed in a separate reaction tube. The amplification products may then be unified for detection. Thus, detection may in some cases occur during amplification, e.g. after or during each cycle of a polymerase chain reaction and/or after the complete amplification reaction.

In one embodiment of the first aspect of the method, the method additionally comprises the step of separating the amplification products by their size or molecular weight before detecting said amplification products. Separation by size or molecular weight can e.g. be performed using electrophoresis, e.g. gel electrophoresis, or chromatographical techniques.

The amplification reaction is preferably selected from the group comprising polymerase chain reaction (PCR), ligase chain reaction (LCR), transcription-based amplification system (TAS), nucleic acid sequence based amplification (NASBA), rolling circle amplification (RCA), transcription-mediated amplification (TMA), self-sustaining sequence replication (3SR), QP amplification and (thermostable) helicase dependent amplification ((t)HAD). More preferably the amplification reaction is a PCR. Most preferably, the amplification reaction is a multiplex PCR, i.e. the amplification of more than one target nucleic acid sequence in a single tube, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 loci (target nucleic acid sequences) may be amplified simultaneously.

As outlined above, the nucleic acid sequences (i.e. the loci) may be amplified simultaneously, e.g. in one reaction tube or separately, e.g. in different reaction tubes. It is preferred in the methods of the present invention that said loci are amplified simultaneously.

Preferably, the amplification reaction is a multiplex PCR.

Preferably, the reaction additionally comprises the step of separating the amplification products by their size or molecular weight before detecting said amplification products.

Preferably, the amplification reaction is real-time multiplex polymerase chain reaction and the method comprises the steps of
(i) simultaneously amplifying at least four loci on nucleic acid templates using pairs of primers substantially complementary to sequences flanking said loci;
(ii) detecting said amplification products using at least one oligonucleotide probe for each locus to be amplified, wherein said oligonucleotide probe is substantially complementary to a sequence on said locus to be amplified,
(iii) wherein a fluorescent dye is covalently attached to each oligonucleotide probe and wherein at least four different dyes are used for differentiating the at least four probes wherein, the dyes may be selected from the group comprising:
   i. a first dye which is 6-FAM or 5-FAM or a blend thereof,
   ii. a second dye which is DY-530, HEX or ATTO 532 or a blend thereof,
   iii. a third dye which is ATT0550 or DY-555 or a blend thereof,
   iv. a fourth dye which is ATT0565, DY510-XL or ROX or a blend thereof,
   v. a fifth dye which is DY632 or DY520-XL or a blend thereof,
   vi. and a sixth dye which is Chromeo 494 and, wherein,
      one dye must be Chromeo 494.

More preferably the amplification reaction is real-time multiplex polymerase chain reaction and the method comprises the steps of:
(i) simultaneously amplifying at least six loci on nucleic acid templates using pairs of primers substantially complementary to sequences flanking said loci;
(ii) detecting said amplification products using at least one oligonucleotide probe for each locus to be amplified, wherein said oligonucleotide probe is substantially complementary to a sequence on said locus to be amplified,
(iii) wherein a fluorescent dye is covalently attached to each oligonucleotide probe and wherein at least six different dyes are used for differentiating the at least six probes wherein, the dyes may be selected from the group comprising:
   i. a first dye which is 6-FAM or 5-FAM or a blend thereof,
   ii. a second dye which is DY-530, HEX or ATTO 532 or a blend thereof,
   iii. a third dye which is ATT0550 or DY-555 or a blend thereof,
   iv. a fourth dye which is ATT0565, DY510-XL or ROX or a blend thereof,
   v. a fifth dye which is DY632 or DY520-XL or a blend thereof,
   vi. and a sixth dye which is Chromeo 494.

Here, one combination is preferred, i.e. 6-FAM, DY-530, ATT0565, ATT0550, DY510-XL, DY632 and Chromeo 494

Ideally, the first dye is 6-FAM, the second dye is ATT0550 the third dye is ATT0565, and the fourth dye is Chromeo 494.

If more than four dyes are used and these are preferably 6-FAM, DY-530, ATT0550, ATT0565, DY510-XL, DY632 and Chromeo 494.

If six dyes are used these are preferably 6-FAM, DY-530, ATT0550, ATT0565, DY632 and Chromeo 494.

If more than six dyes are used these are preferably selected from the group comprising 6-FAM, DY-530, ATT0550, ATT0565, DY510-XL, DY632 and Chromeo 494.

If more than six dyes are used these are preferably selected from the group consisting of 6-FAM, DY-530, ATT0550, ATT0565, DY510-XL, DY632 and Chromeo 494.

Preferably, at least one dye is covalently attached to an oligonucleotide size marker.

The invention also relates to a composition comprising:
(i) a first nucleic acid to which a fluorescent dye selected from the group consisting of 6-FAM and 5-FAM or a blend thereof is covalently attached;
(ii) a second nucleic to which a fluorescent dye selected from the group consisting of ATT0550 and DY-555 is covalently attached;
(iii) a third nucleic acid to which a fluorescent dye selected from the group consisting of ATT0565 and DY510-XL is covalently attached, and
(iv) a fourth nucleic acid to which a fluorescent dye is attached the dye being Chromeo 494.

In a preferred embodiment, the composition comprises:
(i) a first nucleic acid to which a fluorescent dye selected from the group consisting of 6-FAM and 5-FAM or a blend thereof is covalently attached;
(ii) a second nucleic acid to which a fluorescent dye selected from the group consisting of DY-530, HEX or ATTO 532 or a blend thereof is covalently attached;
(iii) a third nucleic to which a fluorescent dye selected from the group consisting of ATT0550 and DY-555 is covalently attached;
(iv) a fourth nucleic acid to which a fluorescent dye selected from the group consisting of ATT0565, DY510-XL and ROX is covalently attached;
(v) a fifth nucleic acid to which a fluorescent dye selected from the group consisting of DY632 or DY520-XL or a blend thereof is covalently attached, and
(vi) a sixth nucleic acid to which a fluorescent dye is attached, the dye being Chromeo 494.

Such a composition may be a pre-mix for a PCR reaction or a multiplex PCR reaction. It may comprise other components as a buffer or an enzyme.

In a further embodiment the invention relates to a kit for multiplex PCR or PCR comprising at least four different fluorescent dyes suitable for the covalent attachment to nucleotides, primers, size markers or oligonucleotide probes used in multiplex PCR, wherein the four dyes may be selected from the six groups below:
i. 6-FAM or 5-FAM or a blend thereof,
ii. DY-530, HEX or ATTO 532 or a blend thereof,
iii. ATT0550 or DY-555 or a blend thereof,
iv. ATT0565, DY510-XL or ROX or a blend thereof,
v. DY632 or DY520-XL or a blend thereof, and
vi. Chromeo 494, wherein
   one dye must be Chromeo 494.

In a further embodiment the invention relates to a kit comprising different dyes, these being:
a. 6-FAM,
b. DY-530,
c. ATT0550,
d. ATT0565
e. DY632 and,
f. Chromeo 494.

In a preferred embodiment, the invention relates to a kit for multiplex PCR or PCR comprising at least six different fluorescent dyes suitable for the covalent attachment to nucleotides, primers, size markers or oligonucleotide probes used in multiplex PCR, wherein the six dyes may be selected from the six groups below:
i. 6-FAM or 5-FAM or a blend thereof,
ii. DY-530, HEX or ATTO 532 or a blend thereof,
iii. ATT0550 or DY-555 or a blend thereof,
iv. ATT0565, DY510-XL or ROX or a blend thereof,
v. DY632 or DY520-XL or a blend thereof, and
vi. Chromeo 494.

In one embodiment the amplification is an amplification of a forensic sample.

### Example

Object of the invention is the use of six defined fluorescent dyes that allow simultaneous detection of PCR products in capillary electrophoresis (CE). The challenge in choosing the fluorescent dyes is dependent on many factors. Forensic labs mainly use CE instruments from ABI (ThermoFisher). The optical setup of these devices consists of a single Argon-(488 nm) or “solide state” diode-laser (505 nm), a spectrograph, virtual filter sets in combination with CCD-cameras and algorithms for spectral calibration, which is entirely different from other CE-instruments or real-time PCR thermocyclers. This specific optical setup strongly limits compatibility of various dyes and thus the repertoire thereof. The aim is to achieve a minimal overlap of emission spectra of six different fluorescent dyes, a higher sensitivity, less crosstalk between the color channels, as well as compatibility with the ABI 3500 CE instrument. A panel of dyes were studied that meet these criteria while matching to the colors of a fluorescence dye combination, which can be used for DNA oligonucleotide labeling and simultaneous detection by DNA sequencing automates, consisting of 6-FAM, DY-530, ATT0550, DY510-XL and DY632. FIG. 1 shows the spectral profile of this matrix standard.

Literature, patent, and internet searches revealed a panel of dyes that, according to the manufacturer, exhibit emission peaks within the defined spectral emission range (peak emission between 592 nm and 641 nm) for the sixth dye (Table 1).

| Name of | Abs Max | Em Max | | Matrix | | Sensi- | |
|---|---|---|---|---|---|---|---|
| Fluorescence Dye | [nm] | [nm] | Source | Setup | Baseline | tivity | Comment |
| ATTO 565 | 563 | 592 | ATTO-TEC | failed | na | na | |
| Dy-590 | 580 | 599 | Dyomics | failed | na | na | |
| ROX, 5-Isomer | 574 | 602 | Biomers | failed | na | na | |
| Atto Rho12 | 576 | 603 | ATTO-TEC | failed | na | na | |
| Texas Red ®-X, 2/4 | 583 | 603 | Biomers | failed | na | na | |
| California Red | 583 | 603 | AAT Bioquest | successful | + | ∘ | |
| Sunnyvale Red | 576 | 605 | AAT Bioquest | successful | ∘ | ∘ | |
| Tide Fluor ™ 4 (TF4) | 588 | 610 | AAT Bioquest | failed | na | na | |
| Atto Rho101 | 586 | 610 | ATTO-TEC | failed | na | na | |
| CAL Fluor RED 610 | 590 | 610 | Biosearch | failed | na | na | |
| CF ™594 | 593 | 614 | Biotium | failed | na | na | |
| ATTO 590 | 594 | 624 | ATTO-TEC | successful | ∘ | reduced | reduced sensitivity |
| ATTO 594 | 601 | 627 | ATTO-TEC | successful | ∘ | reduced | reduced sensitivity |
| Chromeo 494 | 494 | 628 | Active Motif Chromeon | successful | + | + | |
| Dy-610 | 609 | 629 | Dyomics | ∘ | na | reduced | too high amount of PCR product required |
| | | | | | | | for matrix calibration --> indicates low |
| | | | | | | | sensitivity |
| Dy-480 XL | 500 | 630 | Dyomics | successful | + | ∘ | |
| Eterneon | 480 | 635 | Jena Bioscience | failed | na | na | |
| ATTO 620 | 619 | 643 | ATTO-TEC | ∘ | na | reduced | too high amount of PCR product required |
| | | | | | | | for matrix calibration --> indicates low |
| | | | | | | | sensitivity |
| DY-615 | 621 | 641 | Dyomics | ∘ | na | reduced | too high amount of PCR product required |
| | | | | | | | for matrix calibration --> indicates low |
| | | | | | | | sensitivity |

Table 1 shows a list of candidates for the sixth dye as well as the source, feasibility of a CE-matrix with the dye, amount of background signal, and sensitivity. Six candidates could be calibrated successfully, of which five dyes yielded too high background signals or too low signal intensities. Only one dye (Chromeo 494) showed a very low background and strong signal intensity in combination with a five color dyeset, e.g. consisting of 6-FAM, DY-530, ATT0550, ATT0565 and DY632. A high sensitivity of the multiplex PCR assay is achieved by the low background noise and the strong signal intensity. This high sensitivity is a key factor, especially in forensic analyses coping with minuscule amounts of DNA samples.

The crosstalk into the other color channels is minimized by the matching spectrum of the Chromeo 494 with respect to a five color dyeset, e.g. consisting of 6-FAM, DY-530, ATT0550, ATT0565 and DY632. This reduces the occurrence of artefact peaks and facilitates the evaluation and interpretation of results.

This dye combination allows the amplification of more than 20 STR markers in a multiplex PCR and subsequent discrete separation by CE. This results in a distinct separation of single markers while retaining a reduced maximum length of PCR amplicons.

### Figure Captions

FIG. 1: Structures of fluorescence dyes from Dyomics GmbH (Jena, Germany) (A) DY-530 Free carboxylic acid, (B) DY-556 NHS-ester, mono-sodium salt, (C) DY-632 free acid, di-sodium salt, (D) DY-510XL free carboxylic acid, (E) DY-520XL, carboxylic acid. In case of (A), (C), (D) and (E) free carboxyl groups are used for NHS-coupling to the 5′-ends of oligonucleotides.

FIG. 2: Structures of fluorescence dyes from ATTO-TEC GMBH (Siegen, Germany) (A) ATTO 565 (perchlorate) NHS-ester, (B) ATTO 550, free acid of one diasteriomer. The free carboxyl Group is used for NHS-coupling to the 5′-ends of oligonucleotides.

FIG. 3: Structures of fluorescence dye amidires from Biosearch Technologies Inc. (Novato, Calif., USA) (A) CAL Fluor Orange 560, (B) CAL Fluor Red 590, (C) CAL Fluor Red 610, (D) CAL Fluor Red 635. z′Pr: isopropyl, CE: J3-cyanoethyl.

FIG. 4: Shows the absorption and emission spectra of Chromeo 494.

FIG. 5: Structures of fluorescence dye DY-555 (Dyomics GmbH (Jena, Germany)). Structure of NHS-ester of DY-555, chloride salt.

FIG. 6: Spectral profile of a five color dyeset consisting of 6-FAM, DY-530, ATT0550, DY510-XL and DY632. The box marks the free spectrum available for the sixth dye.

FIG. 7 shows the results of a CE matrix calibration with the dye Chromeo 494. In the upper figure spectra of six dyes including Chromeo 494 are shown. The emission peak of Chromeo 494 is positioned exactly between the peaks of the red and orange dye. This allows calibration of six dyes and the general calibration of the matrix. In the lower figure the separation of dye-labelled DNA fragments by size in by is shown. The signal intensities of the six dyes are very similar with respect to each other. The strong signals of Chromeo 494 result in an identical sensitivity of PCR-products in the purple channel in comparison to the other channels. The background signals are very low.

FIG. 8 shows the results of a multiplex PCR in which the forward primers of the systems CSF1PO, D135317 and D5S818 were labelled with Chromeo 494 as the sixth dye. 500 pg human DNA was used as template. The signal intensities of PCR products labelled with Chromeo 494 (purple, last panel) are sufficiently strong (approx. 8000 rfu), are comparable to those of the other channels and enable a high PCR sensitivity.

FIG. 9 shows results of a multiplex-PCR in which the forward primer of the system CSF1PO is labelled with ATTO 594 as the sixth dye. 500 pg human DNA was used as template. The signal intensities of PCR-products labelled with ATTO 594 (purple, last panel) are substantially lower than those of the other channels (approx. 800 rfu to 4000 rfu.

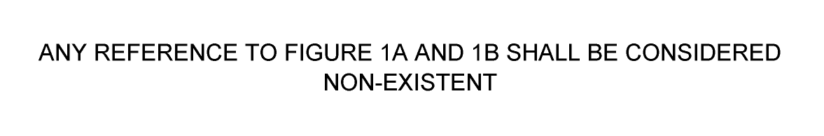

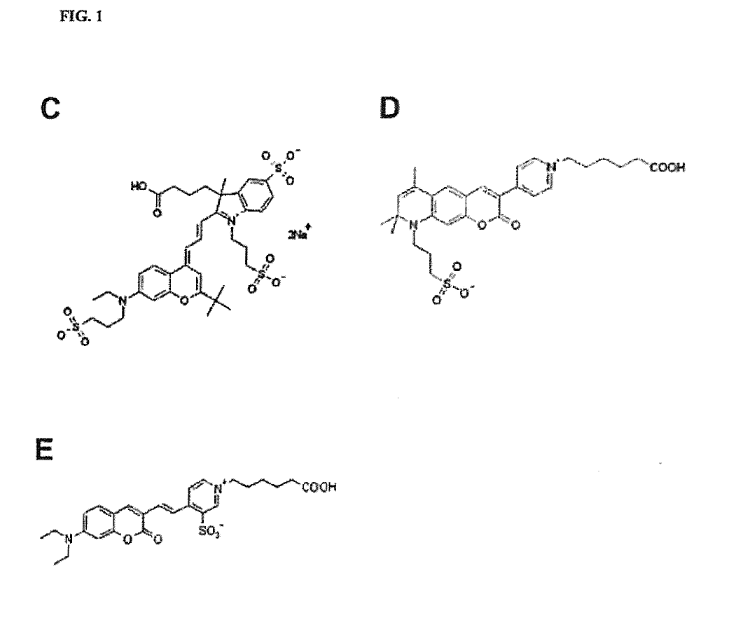

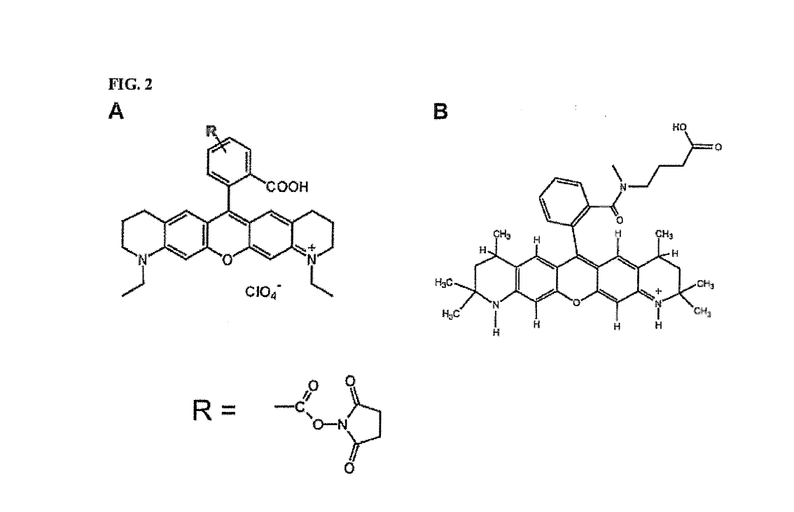

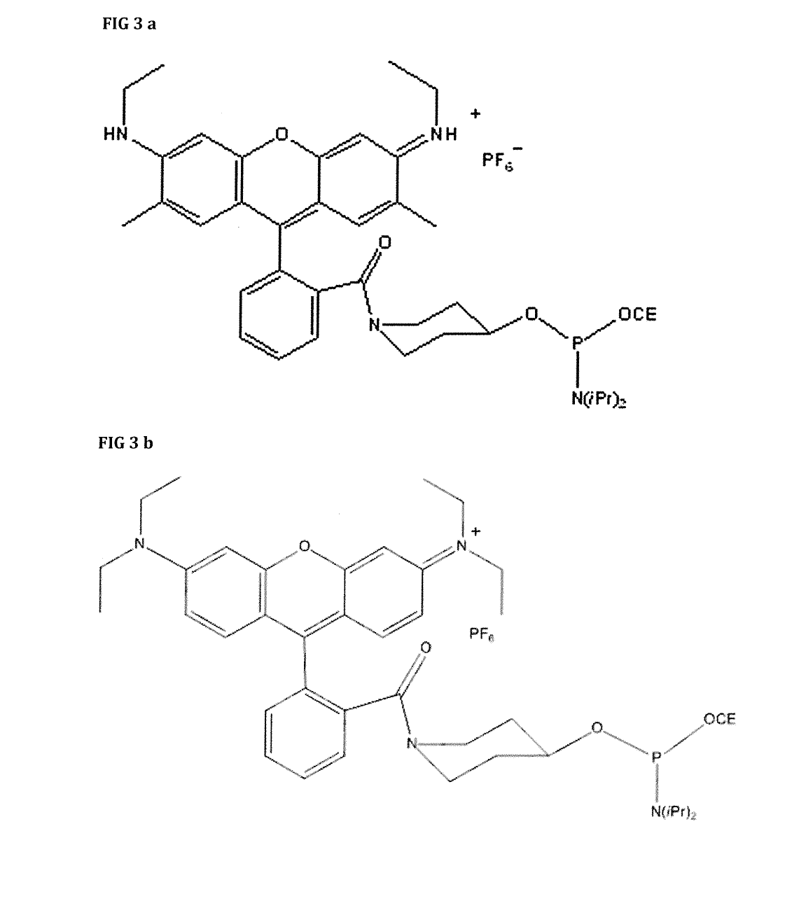

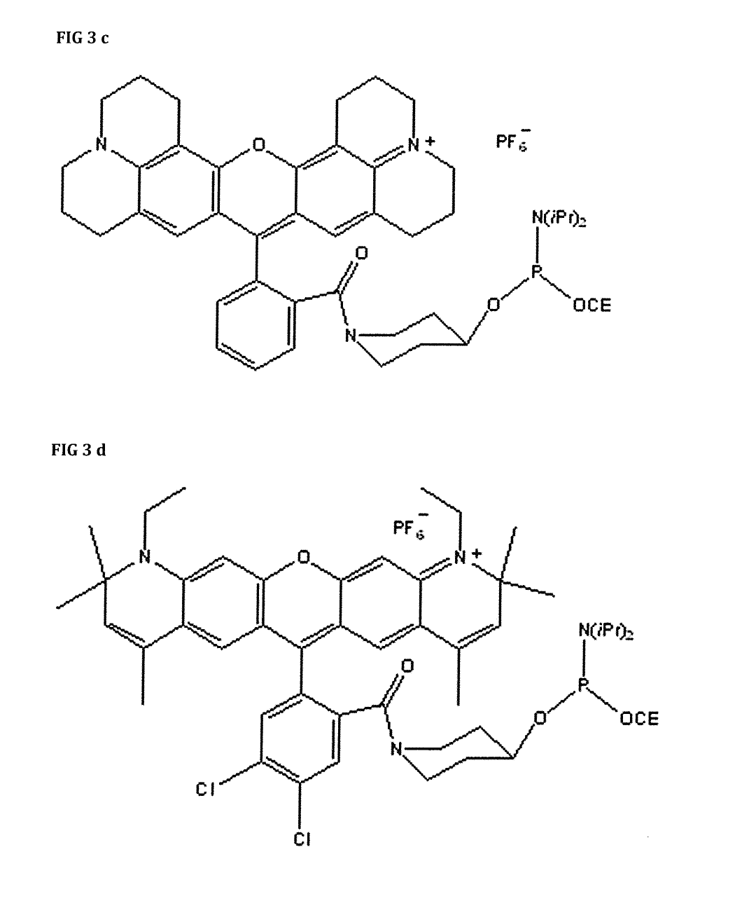

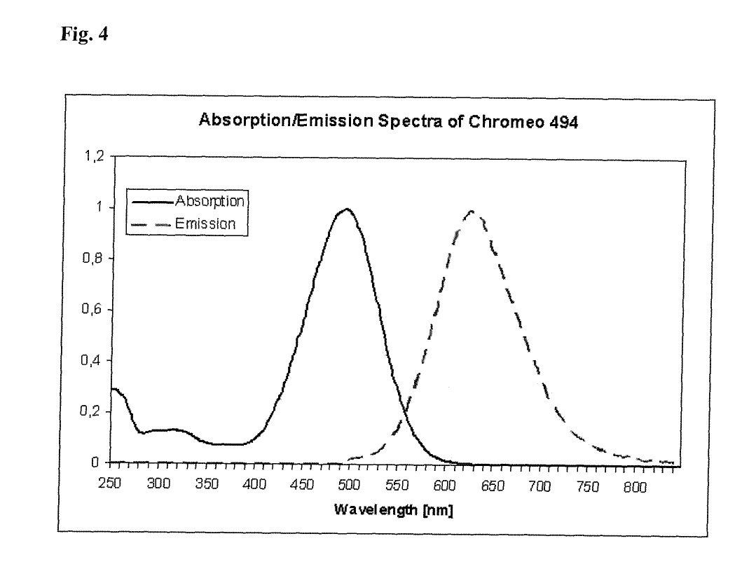

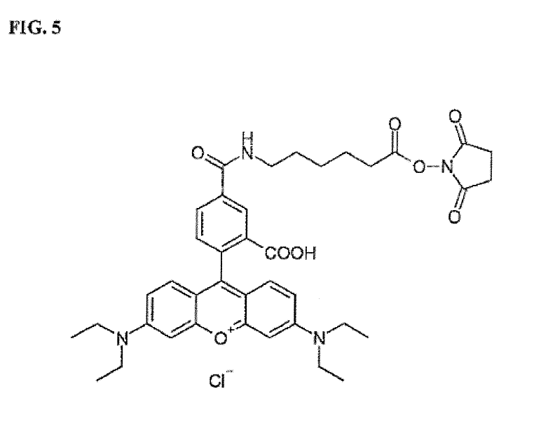

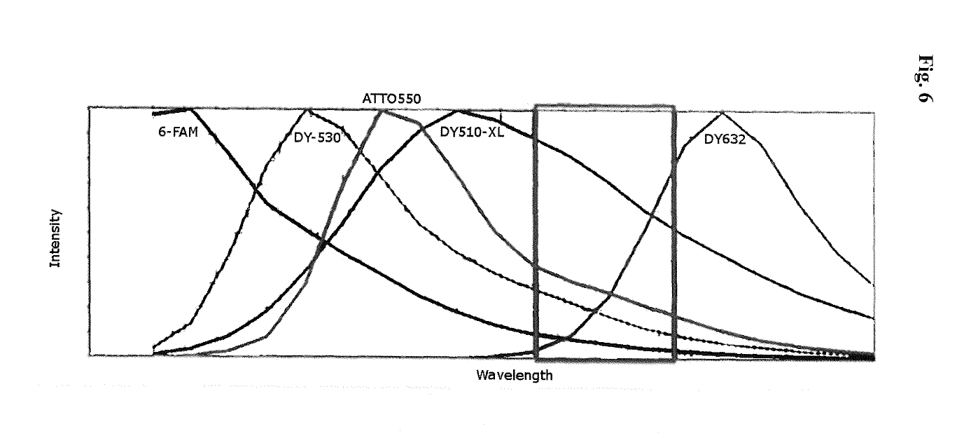

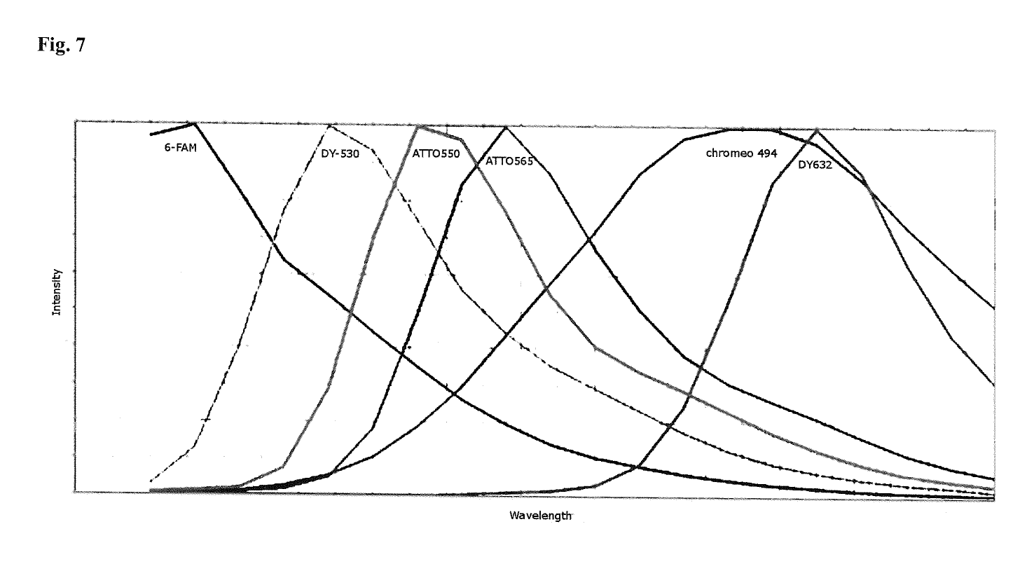

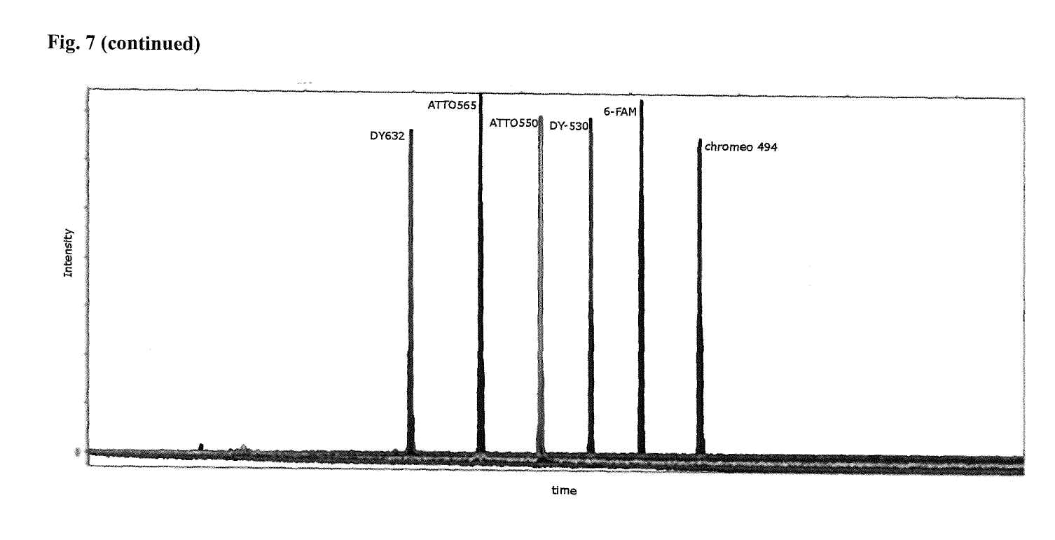

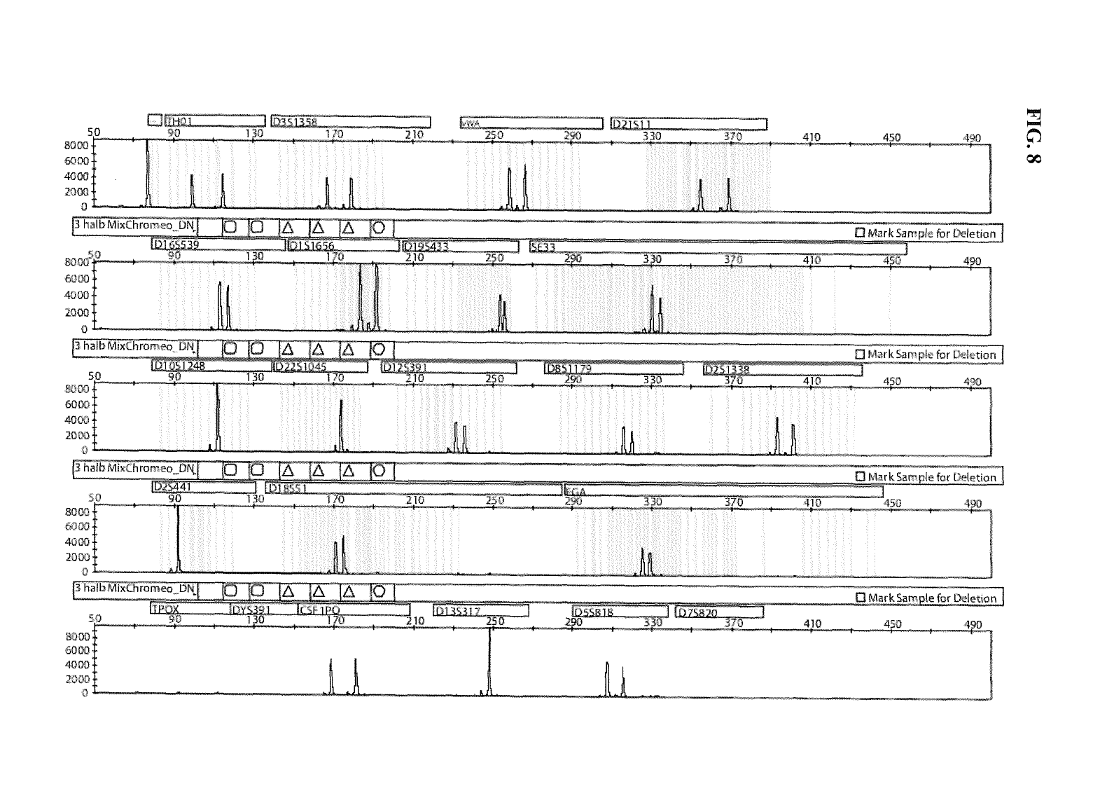

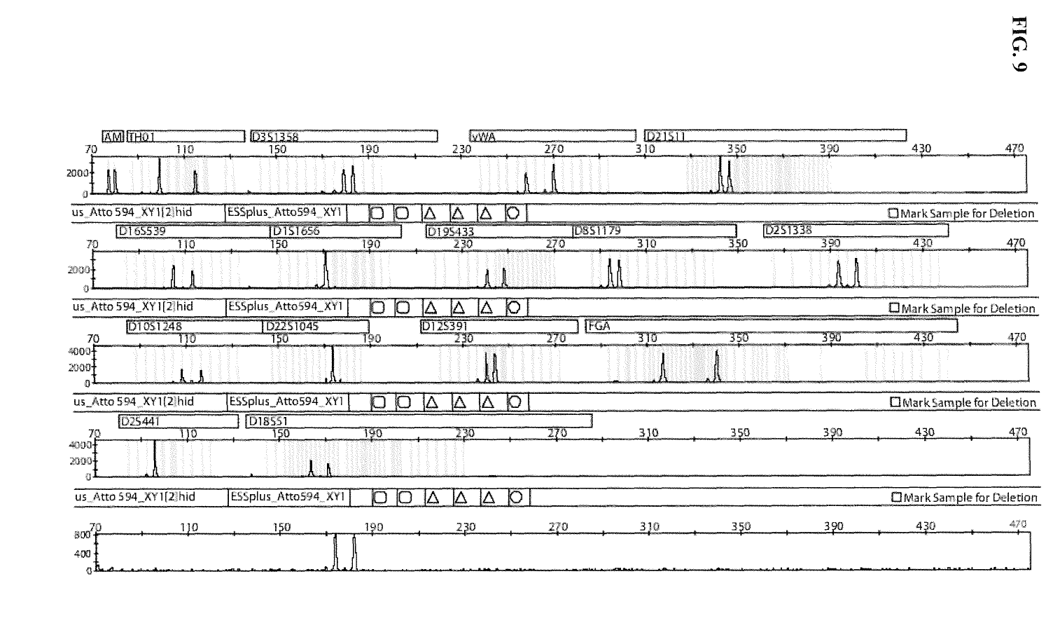

1. Method for the simultaneous detection of at least four nucleic acids labeled with a covalently attached dye in a sample comprising the step of:
detecting the fluorescence emission of said fluorescent dyes upon excitation, wherein the at least four dyes attached to said nucleic acids are selected from the groups of:
i. 6-FAM or 5-FAM or a blend thereof covalently attached to a nucleic acid,
ii. DY-530, HEX or ATTO 532 or a blend thereof covalently attached to a nucleic acid,
iii. ATT0550 or DY-555 or a blend thereof covalently attached to a nucleic acid,
iv. ATT0565, DY510-XL or ROX or a blend thereof covalently attached to a nucleic acid,
v. DY632 or DY520-XL or a blend thereof covalently attached to a nucleic acid and,
vi. Chromeo 494 attached to a nucleic acid, wherein
Chromeo 494 must be one of the four selected.

2. Method for the simultaneous detection of at least four amplification products from an amplification reaction, comprising the steps of
(i) amplifying three or more loci on nucleic acid templates using primers or pairs of primers substantially complementary to sequences flanking said loci; and
(ii) detecting said amplification products by fluorescence detection of fluorescently labelled amplification products or alternatively by fluorescence detection of fluorescently labelled oligonucleotide probes complementary to sequences on said loci of said amplification products,
(iii) wherein a fluorescent dye is covalently attached to said amplification product and/or to said oligonucleotide probe and/or to said primer and/or to at least one primer of said primer pair and optionally to a size marker,
(iv) and wherein at least four different dyes are used for differentiating the at least four amplification products wherein, the dyes may be selected from the group comprising:
i. a first dye which is 6-FAM or 5-FAM or a blend thereof,
ii. a second dye which is DY-530, HEX or ATTO 532 or a blend thereof,
iii. a third dye which is ATT0550 or DY-555 or a blend thereof,
iv. a fourth dye which is ATT0565, DY510-XL or ROX or a blend thereof,
v. a fifth dye which is DY632 or DY520-XL or a blend thereof,
vi. and a sixth dye which is Chromeo 494 and, wherein
one dye must be Chromeo 494.

3. Method according to claim 2, wherein the amplification reaction is selected from the group comprising polymerase chain reaction (PCR), ligase chain reaction (LCR), transcription-based amplification system (TAS), nucleic acid sequence based amplification (NASBA), rolling circle amplification (RCA), transcription-mediated amplification (TMA), self-sustaining sequence replication (3SR), Qβ amplification and (thermostable) helicase dependent amplification ((t)HAD).

4. Method according to claim 2, wherein said loci are amplified simultaneously.

5. Method according to claim 3, wherein the amplification reaction is a multiplex PCR.

6. Method according to claim 2, additionally comprising the step of separating the amplification products by their size or molecular weight before detecting said amplification products.

7. Method according to claim 2, wherein the amplification reaction is real-time multiplex polymerase chain reaction and the method comprises the steps of
(i) simultaneously amplifying at least four loci on nucleic acid templates using pairs of primers substantially complementary to sequences flanking said loci;
(ii) detecting said amplification products using at least one oligonucleotide probe for each locus to be amplified, wherein said oligonucleotide probe is substantially complementary to a sequence on said locus to be amplified,
(iii) wherein a fluorescent dye is covalently attached to each oligonucleotide probe and wherein at least four different dyes are used for differentiating the at least four probes wherein, the dyes may be selected from the group comprising:
i. a first dye which is 6-FAM or 5-FAM or a blend thereof,
ii. a second dye which is DY-530, HEX or ATTO 532 or a blend thereof,
iii. a third dye which is ATT0550 or DY-555 or a blend thereof,
iv. a fourth dye which is ATT0565, DY510-XL or ROX or a blend thereof,
v. a fifth dye which is DY632 or DY520-XL or a blend thereof,
vi. and a sixth dye which is Chromeo 494 and, wherein,
one dye must be Chromeo 494.

8. The method according to claim 1, wherein
a. the first dye is 6-FAM,
b. the second dye is ATT0550,
c. the third dye is ATT0565,
d. and the fourth dye is Chromeo 494.

9. The method according to claim 1, wherein more than four dyes are used and these are:
a. 6-FAM,
b. DY-530,
c. ATT0550,
d. ATT0565
e. DY632 and,
f. Chromeo 494.

10. The method according to claim 2, wherein at least one dye is covalently attached to an oligonucleotide size marker.

11. Composition comprising:
(i) a first nucleic acid to which a fluorescent dye selected from the group consisting of 6-FAM and 5-FAM or a blend thereof is covalently attached;
(ii) a second nucleic to which a fluorescent dye selected from the group consisting of ATT0550 and DY-555 is covalently attached;
(iii) a third nucleic acid to which a fluorescent dye selected from the group consisting of ATT0565, DY510-XL and ROX is covalently attached, and
(iv) a fourth nucleic acid to which a fluorescent dye is attached the dye being Chromeo 494.

12. A kit for multiplex PCR or PCR comprising at least four different fluorescent dyes suitable for the covalent attachment to nucleotides, primers, size markers or oligonucleotide probes used in multiplex PCR, wherein the four dyes may be selected from the six groups below:
i. 6-FAM or 5-FAM or a blend thereof,
ii. DY-530, HEX or ATTO 532 or a blend thereof,
iii. ATT0550 or DY-555 or a blend thereof,
iv. ATT0565, DY510-XL or ROX or a blend thereof,
v. DY632 or DY520-XL or a blend thereof, and
vi. Chromeo 494, wherein
one dye must be Chromeo 494.

13. The kit according to claim 12 comprising at least six different dyes these being:
a. 6-FAM,
b. DY-530,
c. ATT0550,
d. ATT0565
e. DY632 and,
f. Chromeo 494.
